(19) United States
(12) Patent Application Publication
(10) Pub. No.: US 2024/0369559 A1
(43) Pub. Date: Nov. 7, 2024

(54) PANCREATIC CANCER DETECTION

(71) Applicant: RECCAN DIAGNOSTICS AB, Lund (SE)

(72) Inventors: Daniel ANSARI, Lund (SE); Gyorgy MARKO-VARGA, Lund (SE); Roland ANDERSSON, Lund (SE)

(21) Appl. No.: 18/556,559

(22) PCT Filed: Apr. 21, 2022
(86) PCT No.: PCT/EP2022/060631

§ 371 Date: Oct. 20, 2023

Related U.S. Application Data
(60) Provisional application No. 63/201,281, filed on Apr. 21, 2021.

(51) Int. Cl. G01N 33/574; G01N 33/543

(52) U.S. Cl. CPC G01N 33/57438; G01N 33/54388; G01N 2333/47; G01N 2800/06

(57) ABSTRACT

Devices and methods for the detection of biomarkers indicative and/or predictive of pancreatic cancer, wherein the biomarkers are detected in biological samples from a patient. In some examples, a pancreatic cancer detection device may include a solid surface including a plurality of antibodies bound to the solid surface, the solid surface configured to indicate selective binding between each antibody and a corresponding target protein; and wherein the antibodies are configured to selectively bind to two of the target proteins selected from the group consisting of MMP7, CA 19-9, THBS2, MUC2, REG1B, TNC, and TFF1.

### FIELD

This application relates to devices and methods for the detection of biomarkers indicative and/or predictive of pancreatic cancer, wherein the biomarkers are detected in biological samples from a patient.

### BACKGROUND

Pancreatic cancer carries a high mortality rate and is difficult to detect and/or predict. Tremendous efforts have been made to elucidate the mechanisms underlying pancreatic cancer in order to develop effective treatments. Although there have been significant scientific advancements, pancreatic cancer survival rates remain stagnant with a 5-year survival rate of 9%. In the United States, 60,430 patients are predicted to be diagnosed with pancreatic cancer and 48,220 individuals will die from the disease in 2021 (Siegel R L, et al. C A Cancer J Clin; 2021; 71:7-33). Only 10% of patients diagnosed with pancreatic cancer have a localized form treatable by surgical resection; these patients typically have a 5-year survival rate of 39%. Approximately 30% have regional disease associated with a 5-year survival rate of 13%, and the majority of patients (60%) have metastatic pancreatic cancer by the time they have been diagnosed. This carries a 5-year survival rate of just 3%. Despite the continuous overall decline in the death rates from most cancer forms, both incidence and mortality rates for pancreatic cancer have increased during the past decade (Wu W, et al. Clin Epidemiol 2018; 10:789-97). It is projected that pancreatic cancer will become the second leading cause of cancer related death by the year 2030 (Rahib L, et al. Cancer Res 2014; 74 (11): 2913-21).

Surgical resection is the only curative treatment option, yet only about 15-20% of patients are eligible for up-front radical surgery (Kommalapati A, et al. Cancers (Basel) 2018; 10 (1)). Early detection of resectable tumors is key to reduce pancreatic cancer related deaths (Lennon A M, et al. Cancer Res 2014; 74 (13): 3381-9). Late detection of the disease is likely to be the cause for the low survival rate in pancreatic cancer patients with patients not typically identified until late stage. There is an unmet need for a diagnostic tool to be used to identify early-stage pancreatic cancer.

Apart from early diagnosis, molecular markers are needed to accurately predict the course of the disease or response to therapy (Krantz B A et al. Clin Cancer Res 2018; 24 (10): 2241-2250). Carbohydrate antigen 19-9 (CA 19-9 or CA 19.9) is the only clinically approved serum biomarker for pancreatic cancer. However, it suffers from insufficient sensitivity, false negative results in subjects with Lewis-negative genotype (5-10%) and false positivity in several benign conditions. CA 19-9 may be used for disease monitoring in pancreatic cancer patients, but is not recommended for screening purposes (Duffy M J, et al. Ann Oncol 2010; 21 (3): 441-447).

Consequently, to improve patient outcomes, novel and improved diagnostic, prognostic and predictive biomarkers are needed to identify instances of pancreatic cancer and to characterize individual pancreatic tumor biology for the selection of optimal treatment. There is a need for more sensitive and specific assays for pancreatic cancer that could be deployed in near patient settings or at the point of care.

### SUMMARY

Embodiments, examples, and/or aspects of the present disclosure relate to materials, devices, methods, and systems for detection of pancreatic cancer. Some disclosed embodiments, examples, and/or aspects relate to materials, devices, methods, and systems for identifying biomarkers relevant to the detection of pancreatic cancer.

In some examples, a pancreatic cancer detection device may comprise a solid surface comprising a plurality of antibodies bound to the solid surface, the solid surface configured to indicate selective binding between each antibody and a corresponding target protein; and wherein the antibodies are configured to selectively bind to two of the target proteins selected from the group consisting of MMP7, CA 19-9, THBS2, MUC2, REG1B, TNC, and TFF1. The solid surface may comprise antibodies configured to selectively bind three, four, five, six, seven, or more target proteins selected from the group. The solid surface may comprise antibodies configured to selectively bind MMP7, CA 19-9, MUC2, THBS2, and TFF1. The solid surface may comprise antibodies configured to selectively bind MMP7, CA 19-9, TNC, THBS2, and TFF1. The solid surface may comprise antibodies configured to selectively bind MMP7, MUC2, TNC, THBS2, and TFF1. The solid surface may comprise antibodies configured to selectively bind MMP7, MUC2, REG1B, TNC, THBS2, and TFF1.

In certain examples of a pancreatic cancer detection device, the lateral flow detection surface may comprise a lateral flow detection test trip. The pancreatic cancer detection device may further comprise a control line. In the pancreatic cancer detection devices described above, indicating may comprise visually indicating binding to a user.

In some examples, a method of detecting pancreatic cancer may comprise collecting a biological sample from a subject, contacting the biological sample with the pancreatic detection device of any one of the preceding claims, and indicating a likelihood of an incidence of pancreatic cancer in the subject. The biological sample may comprise whole blood, serum, or plasma. The method of detecting pancreatic cancer may further comprise providing a report, the report indicating the likelihood of an incidence of pancreatic cancer in a subject.

In certain aspects, a method for diagnosing pancreatic cancer in a subject may comprise determining levels of at least one, two, three, four, or five markers selected from the group consisting of: Carbohydrate antigen 19-9 (CA 19-9), Cancer antigen 72-4 (CA 72-4), Cancer antigen 125 (CA 125, MUC16), Cancer antigen 242 (CA 242), Carcinoembryonic antigen (CEA), Cartilage oligomeric matrix protein (COMP), Claudin 18 (CLDN18), Complement C2 (C2), Galectin 4 (LGALS4), Gamma-glutamyl transferase 1 (GGT1), Lymphatic vessel endothelial hyaluronan receptor 1 (LYVE1), Matrix metalloproteinase 7 (MMP7), Mucin 2 (MUC2), Mucin 4 (MUC4), Mucin 5AC (MUC5AC), Olfactomedin 4 (OLFM4), Regenerating islet-derived protein 1-alpha (REGIA), Regenerating islet-derived protein 1-beta (REG1B), Serine protease inhibitor Kazal-type 1 (SPINK1), Serpin peptidase inhibitor clade A member 1 (SERPINA1), Syncollin (SYNC), Tenascin C (TNC), Thrombospondin 2 (THBS2), and Trefoil factor 1 (TFF1).

In some aspects, a method for monitoring a subject subjected to surgery or oncological treatment may comprise determining levels of at least one, two, three, four, or five markers in samples taken from the subject at multiple time points including a first sample taken from the subject prior to the treatment to provide baseline levels of the markers and at least one further sample taken from the subject following surgery, wherein the monitored levels of the at least one, two, three, four, or five markers may be used to diagnose or monitor pancreatic cancer following treatment.

Some aspects disclosed herein may relate to a method of determining if a subject has an increased risk of suffering from pancreatic cancer, the method including analyzing at least one sample from the subject to determine levels of individual proteins and comparing the levels of individual proteins with the value of levels of the proteins in one or more normal individuals to determine if the levels of each protein are altered compared to normal levels, wherein a change in the value of the subject's proteins is indicative that the subject has an increased risk of suffering from pancreatic cancer compared to a normal individual.

Alternative or additional embodiments described herein provide a device or apparatus comprising one or more of the features of the foregoing description or of any description elsewhere herein.

Alternative or additional embodiments described herein provide a method or system comprising one or more of the features of the foregoing description or of any description elsewhere herein.

Alternative or additional embodiments described herein provide a lateral flow assay comprising one or more of the features of the foregoing description or of any description elsewhere herein.

Alternative or additional embodiments described herein provide materials, devices, methods, and systems for detecting pancreatic cancer comprising one or more of the features of the foregoing description or of any description elsewhere herein.

### BRIEF DESCRIPTION OF THE DRAWINGS

FIG. 1 depicts an example of a lateral flow detection device.

FIG. 2 depicts a perspective view of an example of another lateral flow detection device.

FIG. 3 depicts an example of a housing for the lateral flow detection device of FIG. 2.

FIG. 4 depicts an example of a methodological workflow for identifying and using biomarkers for the detection of proteins found to be elevated in instances of pancreatic cancer.

FIG. 5 provides examples of a series of graphs depicting the classifier output after analysis of serum samples from patients with and without pancreatic cancer.

FIGS. 6A-D provide examples of analysis of serum samples from patients with and without pancreatic cancer using log regression with backward elimination.

FIGS. 7A-E provide examples of analysis of serum samples from patients with and without pancreatic cancer using a random forest classifier.

### DETAILED DESCRIPTION

Embodiments of the present disclosure relate to materials, devices, methods, and systems for detection of pancreatic cancer. Some disclosed embodiments relate to materials, devices, methods, and systems identification of individual biomarkers or combinations for biomarkers for the detection of pancreatic cancer. One of skill in the art will understand that the materials, devices, methods, and systems may be applied in a general practice and/or clinical setting to patients who are potentially at high risk of developing pancreatic cancer and/or any other suitable patients. One of skill in the art will further understand that such high-risk groups may include patients with family history/genetic risk, new-onset diabetes mellitus after 50 years of age or vague abdominal symptoms (e.g. jaundice, abdominal pain, unexplained weight loss). The materials, devices, methods, and system disclosed herein may be repeated at regular intervals on the same patient and/or across multiple patients to detect and/or predict pancreatic cancer.

It should be noted that throughout the specification the term “comprising” is intended to represent open-ended (i.e. including) language. However, for the avoidance of doubt, wherever the term “comprising” is used it is envisaged that the corresponding feature may also be limited to that specified (i.e. consisting) as necessary.

### Lateral Flow Devices

Provided herein are lateral flow assay devices and methods of using such devices to detect biomarkers for pancreatic cancer in samples from a subject. One of skill in the art will understand that such lateral flow assay devices may be used to detect any of the biomarkers described herein, for example, such lateral flow assay devices may be used to detect any suitable combination of biomarkers described herein and provide an indication of an instance of pancreatic cancer. Further, although lateral flow devices are described in detail herein, one of skill in the art will understand that other types of devices and systems may be suitable for the detection of pancreatic cancer, therefore this disclosure is not limited to the use of a lateral flow device. Further information regarding lateral flow devices may be found in PCT Patent Application WO2017163087A1, the disclosure of which is incorporated by reference herein in its entirety.

The term “immobilized” or “embedded” interchangeably refers to reversibly or irreversibly immobilized molecules (e.g., analytes or binding agents). In some examples, reversibly immobilized molecules are immobilized in a manner that allows the molecules, or a portion thereof (e.g., at least about 25%, 50%, 60%, 75%, 80% or more of the molecules), to be removed from their immobilized location without substantial denaturation or aggregation. For example, a molecule can be reversibly immobilized in or on an absorbent material (e.g., an absorbent pad) by contacting a solution containing the molecule with the absorbent material, thereby soaking up the solution and reversibly immobilizing the molecule. The reversibly immobilized molecule can then be removed by wicking the solution from the absorbent material, or from one region of the absorbent material to another. In some cases, a molecule can be reversibly immobilized on an absorbent material by contacting a solution containing the molecule with the absorbent material, thereby soaking up the solution, and then drying the solution-containing absorbent material. The reversibly immobilized molecule can then be removed by contacting the absorbent material with another solution of the same or a different composition, thereby solubilizing the reversibly immobilized molecule, and then wicking the solution from the absorbent material, or from one region of the absorbent material to another.

Irreversibly immobilized molecules (e.g., binding agents or analytes) are immobilized such that they are not removed, or not substantially removed, from their location under mild conditions (e.g., pH between about 4-9, temperature of between about 4-65° C.). Exemplary irreversibly immobilized molecules include protein analytes or binding agents bound to a nitrocellulose, polyvinylidene fluoride, nylon or polysulfone membrane by standard blotting techniques (e.g., electroblotting). Other exemplary irreversibly immobilized molecules include protein analytes or binding agents bound to glass or plastic (e.g., a microarray, a microfluidic chip, a glass histology slide or a plastic microtiter plate having wells with bound protein analytes therein).

The term “binding agent” refers to an agent that specifically binds to a molecule such as an analyte. While antibodies are described in many contexts herein, it will be understood by one of skill in the art that other binding agents can be used instead of antibodies as preferred by the user. A wide variety of binding agents are known in the art, including antibodies, aptamers, affimers, lipocalins (e.g., anticalins), thioredoxin A, bilin binding protein, or proteins containing an ankyrin repeat, the Z domain of staphylococcal protein A, or a fibronectin type III domain. Other binding agents include, but are not limited to, biotin/streptavidin, chelating agents, chromatography resins, affinity tags, or functionalized beads, nanoparticles and magnetic particles.

The term “specifically bind” refers to a molecule (e.g., binding agent such as an antibody or antibody fragment) that binds to a target with at least 2-fold greater affinity than non-target compounds, e.g., at least about 4-fold, 5-fold, 6-fold, 7-fold, 8-fold, 9-fold, 10-fold, 20-fold, 25-fold, 50-fold, 100-fold, 1000-fold, or more than 1000-fold greater affinity.

The term “antibody” refers to a polypeptide comprising a framework region from an immunoglobulin gene, or fragments thereof, that specifically bind and recognize an antigen, e.g., a particular analyte. Typically, the “variable region” contains the antigen-binding region of the antibody (or its functional equivalent) and is most critical in specificity and affinity of binding. Antibodies include for example chimeric, human, humanized antibodies, or single-chain antibodies.

An exemplary immunoglobulin (antibody) structural unit comprises a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (VL) and variable heavy chain (VH) refer to these light and heavy chains respectively.

Antibodies can exist as intact immunoglobulins or as any of a number of well-characterized fragments that include specific antigen-binding activity. Such fragments can be produced by digestion with various peptidases. Pepsin digests an antibody below the disulfide linkages in the hinge region to produce F (ab)′2, a dimer of Fab which itself is a light chain joined to VH-CH1 by a disulfide bond. The F (ab)′2 may be reduced under mild conditions to break the disulfide linkage in the hinge region, thereby converting the F (ab)′2 dimer into an Fab′ monomer. The Fab′ monomer is essentially Fab with part of the hinge region (see Fundamental Immunology (Paul ed., 3d ed. 1993). While various antibody fragments are defined in terms of the digestion of an intact antibody, one of skill will appreciate that such fragments may be synthesized de novo either chemically or by using recombinant DNA methodology. Thus, the term antibody, as used herein, also includes antibody fragments either produced by the modification of whole antibodies, or those synthesized de novo using recombinant DNA methodologies (e.g., single chain Fv) or those identified using phage display libraries.

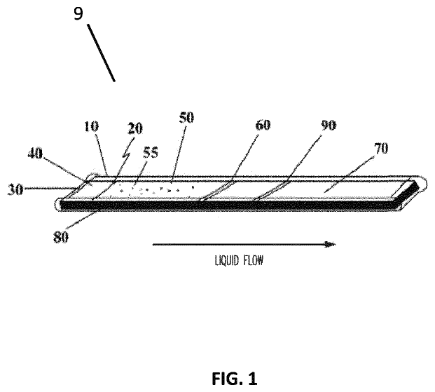

An example lateral flow device 9 for detecting pancreatic cancer is shown in FIG. 1, which shows a schematic diagram of a device with an elongated housing 10 that contains a lateral flow strip 20. The lateral flow strip 20 may extend substantially the entire length of housing 10. The lateral flow strip 20 may be divided into a sample application area 40 positioned below an optional sample introduction port 30, an antigen-antibody conjugation site 50, a capture area 60, and a distal absorbent pad 70. The antigen-antibody conjugation site 50 can have mobile antigens 55. The flow strip 20 can also have a backing 80. The mobile antigen 55 in the antigen-antibody conjugation site 50 can be labeled antigens (such as gold-conjugated antigen) that can react with and bind to antibodies in a test sample from a subject. A flow path along the lateral flow strip 20 passes from the sample application area 40, through the antigen-antibody conjugation site 50, into the capture area 60. Immobilized binding entities such as one or more antibodies that recognize one or more proteins correlated with pancreatic cancer, are positioned on capture area 60. Alternatively, the mobile antigens 55 can bind one or more antibodies that may be present in a test sample and the liquid flow can transport a conjugate formed between a mobile antigen and an antibody to the capture area 60, where immobilized binding entities can capture the antigen-antibody conjugates and concentrate the label in the capture area 60. The mobile antigens 55 without a bound antibody pass through the capture area 60 and are eventually collected in the distal absorbent pad 70. The lateral flow strip 20 can also include a reaction verification or control area 90. Such a control area 90 (e.g., configured as line) can be slightly distal to the capture area 60. The reaction verification or control area 90 illustrates to a user that the test has been performed. Prior to the test being performed, the reaction verification or control area 90 is not visible. However, when the test is performed by placing a fluid sample on the sample application area 40, the reaction verification or control area 90 can become visible as the sample flows through the capture area 60 and to the distal absorbent pad 70. For example, the reaction verification or control area 90 can become visible due to a chemical reacting with any component of the sample or simply due to the presence of moisture in the sample.

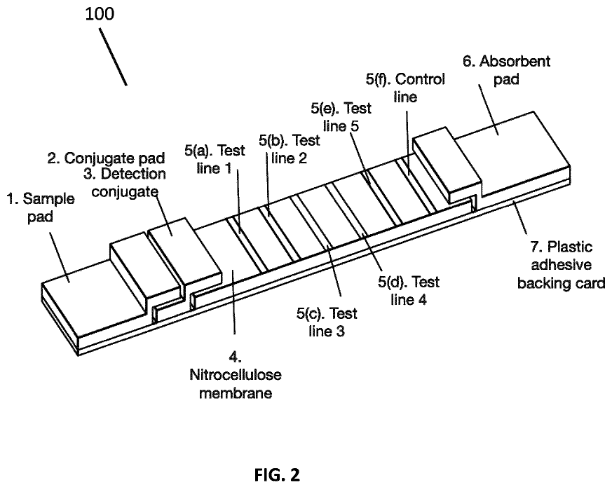

FIG. 2 depicts an example of a lateral flow device 100 for detecting pancreatic cancer, similar to the lateral flow device as depicted above in FIG. 1. The lateral flow device 100 may include a sample pad 1 to which a diluted or neat sample (such as blood, serum, plasma, or other suitable substance) may be added. The lateral flow device 100 may also include a conjugate pad 2 containing an optimized mixture of gold colloid-conjugated antibodies 3 or other suitable detection conjugate for interaction with the test line(s) 5a-5 and control line 5f. One of skill in the art will understand that the lateral flow device 100 may contain any suitable number of test lines, each test line corresponding to one of more antibodies which bind to various analytes in the sample (such as biomarkers). For example, the lateral flow device may have 5 test lines as shown in FIG. 2, or may have 1, 2, 3, 4, 6, 7, 8, 9, 10, 11, 12, or more test lines as needed. One of skill in the art will further understand that each test line may have 1, 2, 3, 4, or more different antibodies configured to bind to various analytes.

Similar to the lateral flow device of FIG. 1, in use a sample containing unknown concentrations of one or more analytes (which include biomarkers) may be placed on the sample pad 1. One of skill in the art will understand that such a sample may be in the form of whole blood (such as from a finger prick capillary), serum, plasma, or any suitable substance. The analyte may then flow along the lateral flow device binding with the detection conjugate and travel along the nitrocellulose membrane 4 towards the test lines 5a-e. The test lines 5a-e each consist of an antibody match to the respective target analyte and, if present in the sample, will form a complex resulting in a visible test line. The control line 5f may consist of an antigen to the control line antibody conjugated to gold, for example BSA-biotin to anti-biotin gold, thereby informing the user that the test has run successfully. In some embodiments all lines may be quantifiable within a certain time period with a suitable lateral flow device reader capable of reading all test lines. The time period may be from about: 1 to 20 minutes, 2 to 18 minutes, 4 to 16 minutes, 6 to 14 minutes, 8 to 12 minutes, or around 10 minutes. The lateral flow device may further be supported by a plastic adhesive backing card.

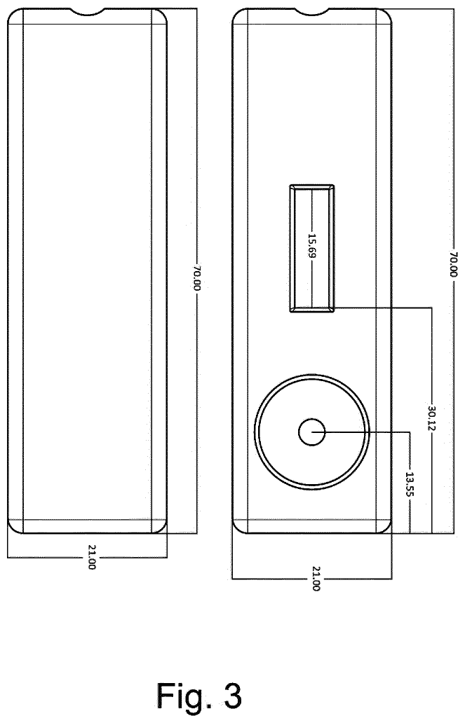

FIG. 3 depicts an example of a housing for a lateral flow device, such as the devices of FIGS. 1-2. An example of measurements are shown in FIG. 3 are in mm. In certain examples, the housing may include a location for applying a test sample, and a window for reading the results. The length of the device may range from about 50 to 100m, 60 to 90 mm, 70-80 mm or 75 mm. The width may range from about 10 to 50 mm, such as 20 to 40 mm, or about 21 mm. The sample application location may be about 10 to 30 mm from one end of the device, or about 13.55 mm from one end. The window may be positioned about 10-40 mm, or about 30 mm from the end of the housing.

Using the methods described herein, one or more lateral flow device(s) or other suitable devices may be used to detect one or more protein biomarkers associated with pancreatic cancer described in much greater detail below, selected from the group consisting of Carbohydrate antigen 19-9 (CA 19-9), Carcinoembryonic antigen (CEA), Cartilage oligomeric matrix protein (COMP), Claudin 18 (CLDN18), Complement C2 (C2), Galectin 4 (LGALS4), Gamma-glutamyl transferase 1 (GGT1), Lymphatic vessel endothelial hyaluronan receptor 1 (LYVE1), Matrix metalloproteinase 7 (MMP7), Mucin 2 (MUC2), Mucin 4 (MUC4), Olfactomedin 4 (OLFM4), Regenerating islet-derived protein 1-alpha (REGIA), Regenerating islet-derived protein 1-beta (REG1B), Serine protease inhibitor Kazal-type 1 (SPINK1), Serpin peptidase inhibitor clade A member 1 (SERPINA1), Syncollin (SYNC), Tenascin C (TNC), Thrombospondin 2 (THBS2), and Trefoil factor 1 (TFF1). Either the complete set of these proteins (20 proteins) or a subset thereof may be detected. A subset may include any combination of 2, 3, 4, 5, 6, 7, 8, 9, 10, 11, 12, 13, 14, 15, 16, 17, 18, or 19 of these proteins. As will be explained in further detail below, these biomarkers have been identified as potential indicators for the detection of pancreatic cancer.

The larger group of 20 biomarkers identified above were selected based upon database searching, literature review, and experimental research to identify a starting template of secreted or cell surface-associated membrane proteins that had been found to be elevated in pancreatic cancer. As will be described in further detail below, these 20 biomarkers were then tested for in serum and classifier algorithmic techniques were used to identify key combinations for the detection of pancreatic cancer. The 20 biomarkers were selected from a larger group of 17165 unique proteins from normal and malignant human tissues. Multiple sources were used to identify these 20 biomarkers, which were identified by narrowing down toward secreted and cell surface-associated proteins that are overexpressed in pancreatic cancer tissue. Uhlen M, et al. (2015) Proteomics. Tissue-based map of the human proteome. Science 347:1260419; Zhou Q, et al. EBioMedicine 2019; 43:282-294; Uhlen M, et al. Proteomics. Tissue-based map of the human proteome. Science 2015; 347:1260419 (the tissue atlas).

In certain examples, as explained in further detail below, the lateral flow assay may include a combination of 5 biomarkers, such as CA 19-9, TFF1, THBS2, MMP7 and MUC2. In some examples, the 5 biomarkers may include a combination of CA 19-9, TFF1, THBS2, MMP7 and TNC. In some examples, the lateral flow assay may include a combination of six biomarkers, such as MMP7, MUC2, REG1B, TNC, THBS2, and TFF1. The lateral flow assay may include a combination of seven biomarkers, such as CA 19.9, MMP7, MUC2, REG1B, TNC, THBS2, and TFF1. The combination of biomarkers may also be a four marker combination, such as TFF1, THBS2, MMP7 and MUC2. The combination of biomarkers may also be a 2 marker combination of MMP7 and MUC2. One of skill in the art will understand that the biomarkers may be organized in any suitable order amongst the test lines on the lateral flow device.

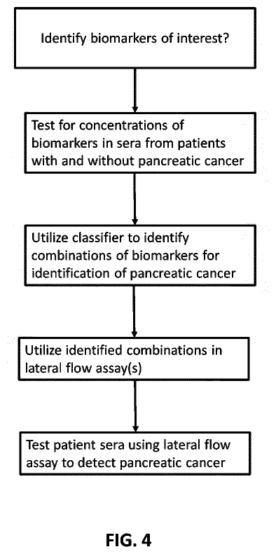

One of skill in the art will understand that there are multiple development pathways that may be followed to develop a lateral flow assay for the detection of pancreatic cancer. For example, FIG. 4 depicts a flowchart showing example overall steps to use classifier algorithms to analyze a set of data and identify combinations of biomarkers to be used in the detection of pancreatic cancer. First, identify a set of biomarkers overexpressed in pancreatic cancer tissue with a focus on secreted and cell surface-associated proteins. The concentrations of these biomarkers are then tested on sera collected from patients with and without pancreatic cancer, such as patients with advanced, unresectable pancreatic cancer and patients with early stage, resectable pancreatic cancer. Next, a classifier may be used to identify combinations of proteins for identification of pancreatic cancer. These identified combinations can then be utilized in lateral flow assays or other suitable assays to detect pancreatic cancer. Once pancreatic cancer is detected, then confirmation via other techniques (such as via CT scan) may be necessary. Once the pancreatic cancer is confirmed, then a patient may receive appropriate treatment, such as surgery or other suitable techniques.

In some examples, patients may be monitored via the lateral flow assay devices described herein, such as once a year during an annual checkup, every 2 years, every 3 years, every 5 years, or longer than 5 years. Patients may be monitored multiple times per year, such as twice per year, 3 times per year, 4 times per year, or more than 4 times per year.

One of skill in the art will further understand that after a positive indication of possible detection of pancreatic cancer via the lateral flow assay devices described herein, the presence of pancreatic cancer may be confirmed by another test, such as a CT scan or other suitable device. Additionally, one of skill in the art will understand that the combinations of biomarkers described herein for detecting pancreatic cancer may also be analyzed via other methods, instead of via lateral flow assay. For example, they may be analyzed via a multiplex assay such as a Luminex platform, use of enzyme-linked immunosorbent assay (ELISA) techniques, chromatography techniques, and/or other suitable protein detection techniques.

### Biomarker Combinations

In an example method to identify biomarkers and biomarker combinations that may be used to identify pancreatic cancer, such as via a lateral flow assay as described above, serum samples were prospectively collected from 400 individuals, including 100 patients with pancreatic ductal adenocarcinoma, 100 patients with benign pancreatic and hepatobiliary disease, as well as 200 age and gender-matched healthy controls between 2014 and 2022. Patient sera were obtained at diagnosis, prior to treatment at Skane University Hospital, Lund, Sweden. Of the 100 pancreatic cancer patients, 50 patients had early-stage, resectable disease, while 50 patients had advanced, unresectable disease. Healthy control sera were obtained from donors at the blood donation center in Lund, Sweden. Blood samples were collected in BD SST II Advance tubes. The minimum clotting time was 30 minutes. The samples were centrifuged at 2000×g for 10 minutes at 25° C., and serum was collected and stored at −80° C. until further analysis.

To select which biomarkers should be used alone or in combination to detect pancreatic cancer, the concentration of the twenty biomarkers shown below in Table 1 were measured in the patient sera. The identification of this particular group of 20 biomarkers is described elsewhere herein. Serum samples were stored at −80° C. until the day of testing. On the day of testing the appropriate samples were removed from −80° C. storage and allowed to thaw at room temperature for 1 hour. Biomarker levels in serum samples were measured via ELISA according to manufacturer's instructions. ELISA assays are well known in the art and further details may be found in in PCT Patent Application WO2017163087A1, the disclosure of which is incorporated by reference herein in its entirety. Serum samples were stored at −80° C. until the day of testing. On the day of testing the appropriate samples were removed from −80° C. storage and allowed to thaw at room temperature for 1 hour.

TABLE 1
Biomarkers of interest for detecting pancreatic cancer

| Item | | |
|---|---|---|
| description | Biomarker | Provider |
| ELISA Kit | Carbohydrate antigen 19-9 | Monobind |
| | (CA 19-9) | |
| ELISA Kit | Cancer antigen 72-4 (CA 72-4) | Tecan |
| ELISA Kit | Cancer antigen 125 (CA 125, | Monobind |
| | MUC16) | |
| ELISA Kit | Cancer antigen 242 (CA 242) | Fujirebio |
| ELISA Kit | Carcinoembryonic antigen (CEA) | Monobind |
| ELISA Kit | Cartilage oligomeric matrix | BioTechne |
| | protein (COMP) | |
| ELISA Kit | Claudin 18 (CLDN18) | Abclonal |
| ELISA Kit | Complement C2 (C2) | Novus |
| ELISA Kit | Galectin 4 (LGALS4) | Novus |
| ELISA Kit | Gamma-glutamyl transferase 1 | Cloud Clone |
| | (GGT1) | |
| ELISA Kit | Lymphatic vessel endothelial | Ray Biotech |
| | hyaluronan receptor 1 (LYVE1) | |
| ELISA Kit | Matrix metalloproteinase 7 | BioTechne |
| | (MMP7) | |
| ELISA Kit | Mucin 2 (MUC2) | Novus |
| ELISA Kit | Mucin 4 (MUC4) | Cloud Clone |
| ELISA Kit | Mucin 5AC (MUC5AC) | LSBio |
| ELISA Kit | Olfactomedin 4 (OLFM4) | Cloud Clone |
| ELISA Kit | Regenerating islet-derived | Cloud Clone |
| | protein 1-alpha (REG1A) | |
| ELISA Kit | Regenerating islet-derived | Cloud Clone |
| | protein 1-beta (REG1B) | |
| ELISA Kit | Serine protease inhibitor | Cloud Clone |
| | Kazal-type 1 (SPINK1) | |
| ELISA Kit | Serpin peptidase inhibitor | Mologic |
| | clade A member 1 (SERPINA1) | |
| ELISA Kit | Syncollin (SYNC) | Cloud Clone |
| ELISA Kit | Tenascin C (TNC) | Immune- |
| | | Biological Lab (IBL) |
| ELISA Kit | Thrombospondin 2 (THBS2) | Abcam, Abbexa |
| ELISA Kit | Trefoil factor 1 (TFF1) | Cloud Clone |

Continuing with the example, the raw ELISA results were run through a classifier algorithm to identify combinations of biomarkers that provide an indication of pancreatic cancer. As will be understood by one of skill in the art, a classifier is a software algorithm that automatically orders or categorizes data into one or more sets of classes. As will further be understood by one of skill in the art, a classifier may be trained by data sets such that the classifier may classify data into the desired classes. In the example discussed here, the classifier was trained from the raw ELISA results, using combinations of biomarkers (such as five) from Table 1. The classifier may be used to identify a combination of up to 5 biomarkers (including CA 19-9) that may provide improved detection of pancreatic cancer over CA 19-9. As described above, CA 19-9 is used alone as the current standard biomarker for pancreatic cancer detection. However, as also explained above, CA 19-9 alone suffers from insufficient sensitivity, false negative results in subjects with Lewis-negative genotype (5-10%) and false positivity in several benign conditions.

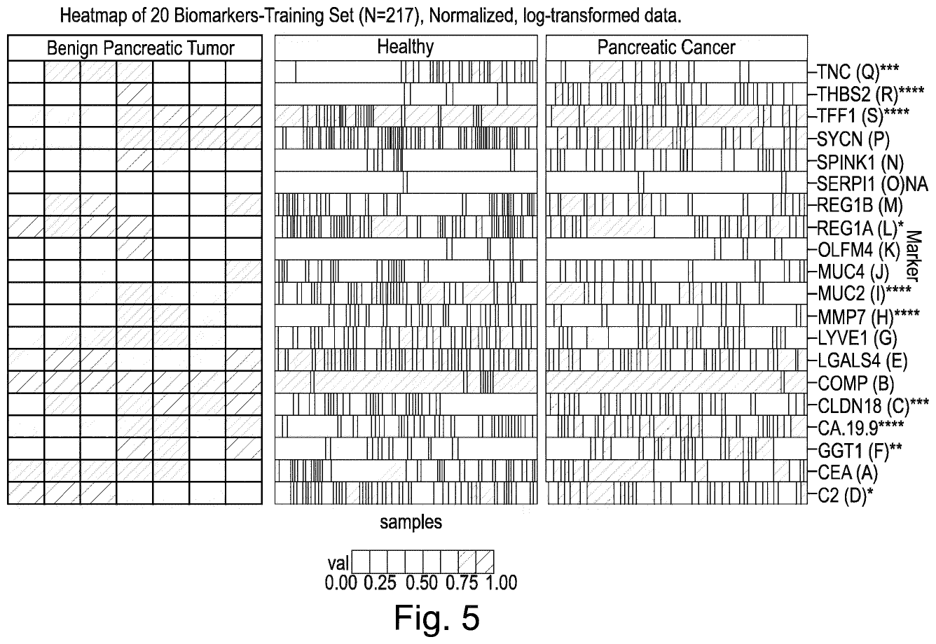

Continuing with the example, the raw ELISA results were split 70% into a training data set and 30% into a holdout set. The training set was used to determine distribution of the markers and to transform the marker data, perform variable (predictor) selection and model tuning, and perform threshold optimization. Two models were applied, Lasso and LDA. Table 2 below shows the accuracy result for detection of pancreatic cancer as determined by the classifier. Area under the curve (AUC) provides a calculation of the area under the curve in a graphed plot of the data when sensitivity is on the Y axis and specificity is on the X axis. Therefore, a higher AUC is generally more desirable, indicating a better combination of biomarkers for the detection of pancreatic cancer. As shown in the table, the combination of the biomarkers CA 19-9, TFF1, THBS2, MMP7 and MUC2 unexpectedly provided both improved sensitivity and improved specificity for the detection of pancreatic cancer over the use of CA 19-9 alone. Similarly, the combination of biomarkers CA 19-9, TFF1, THBS2, MMP7 and TNC also unexpectedly provided both improved sensitivity and improved specificity for the detection of pancreatic cancer over the use of CA 19-9 alone. FIG. 5 depicts the sensitivity versus specificity graphs for CA 19.9, MMP7, MUC2, TFF1, THBS2, SERPINA1, TNC, CLDN18, and SPINK1. As shown in the graphs, the AUC, sensitivity and specificity of CLDN18 and SPINK1 tended to lag behind the other biomarkers.

TABLE 2
Accuracy results for combinations of biomarkers
(blind dataset), Pancreatic Cancer vs Healthy

| | | | | |
|---|---|---|---|---|
| threshold | sensitivity | specificity | ppv | npv |

A) MMP7 + CA.19.9 + MUC2 + THBS2 + TFF1

| | | | | |
|---|---|---|---|---|
| 0.20 | 100.00000 | 86.84211 | 80.76923 | 100.00000 |
| 0.25 | 100.00000 | 89.47368 | 84.00000 | 100.00000 |
| 0.35 | 95.23810 | 97.36842 | 95.23810 | 97.36842 |
| 0.40 | 90.47619 | 97.36842 | 95.00000 | 94.87179 |
| 0.45 | 90.47619 | 97.36842 | 95.00000 | 94.87179 |
| 0.50 | 90.47619 | 97.36842 | 95.00000 | 94.87179 |

B) CA.19.9 + MMP7 + MUC2 + REG1B + TNC + THBS2 + TFF1

| | | | | |
|---|---|---|---|---|
| 0.20 | 100.00000 | 84.21053 | 77.77778 | 100.00000 |
| 0.25 | 100.00000 | 84.21053 | 77.77778 | 100.00000 |
| 0.35 | 95.23810 | 89.47368 | 83.33333 | 97.14286 |
| 0.40 | 95.23810 | 89.47368 | 83.33333 | 97.14286 |
| 0.45 | 95.23810 | 92.10526 | 86.95652 | 97.22222 |
| 0.50 | 95.23810 | 92.10526 | 86.95652 | 97.22222 |

C) MMP7 + MUC2 + REG1B + TNC + THBS2 + TFF1

| | | | | |
|---|---|---|---|---|
| 0.20 | 100.00000 | 86.84211 | 80.76923 | 100.00000 |
| 0.25 | 100.00000 | 86.84211 | 80.76923 | 100.00000 |
| 0.35 | 100.00000 | 89.47368 | 84.00000 | 100.00000 |
| 0.40 | 100.00000 | 89.47368 | 84.00000 | 100.00000 |
| 0.45 | 100.00000 | 89.47368 | 84.00000 | 100.00000 |
| 0.50 | 100.00000 | 89.47368 | 84.00000 | 100.00000 |

D) MMP7 + MUC2 + TNC + THBS2 + TFF1

| | | | | |
|---|---|---|---|---|
| 0.20 | 100.00000 | 89.47368 | 84.00000 | 100.00000 |
| 0.25 | 100.00000 | 89.47368 | 84.00000 | 100.00000 |
| 0.35 | 95.23810 | 92.10526 | 86.95652 | 97.22222 |
| 0.40 | 95.23810 | 92.10526 | 86.95652 | 97.22222 |
| 0.45 | 95.23810 | 92.10526 | 86.95652 | 97.22222 |
| 0.50 | 95.23810 | 92.10526 | 86.95652 | 97.22222 |

E) MMP7 + CA.19.9 + MUC2 + THBS2 + TFF1

| | | | | |
|---|---|---|---|---|
| 0.20 | 100.00000 | 86.84211 | 80.76923 | 100.00000 |
| 0.25 | 100.00000 | 89.47368 | 84.00000 | 100.00000 |
| 0.35 | 95.23810 | 97.36842 | 95.23810 | 97.36842 |
| 0.40 | 90.47619 | 97.36842 | 95.00000 | 94.87179 |
| 0.45 | 90.47619 | 97.36842 | 95.00000 | 94.87179 |
| 0.50 | 90.47619 | 97.36842 | 95.00000 | 94.87179 |

F) MMP7 + MUC2 + THBS2 + TFF1

| | | | | |
|---|---|---|---|---|
| 0.20 | 100.00000 | 84.21053 | 77.77778 | 100.00000 |
| 0.25 | 95.23810 | 89.47368 | 83.33333 | 97.14286 |
| 0.35 | 95.23810 | 92.10526 | 86.95652 | 97.22222 |
| 0.40 | 95.23810 | 94.73684 | 90.90909 | 97.29730 |
| 0.45 | 95.23810 | 94.73684 | 90.90909 | 97.29730 |
| 0.50 | 90.47619 | 94.73684 | 90.47619 | 94.73684 |

G) MMP7 + MUC2 + THBS2

| | | | | |
|---|---|---|---|---|
| 0.20 | 100.00000 | 84.21053 | 77.77778 | 100.00000 |
| 0.25 | 100.00000 | 84.21053 | 77.77778 | 100.00000 |
| 0.35 | 95.23810 | 86.84211 | 80.00000 | 97.05882 |
| 0.40 | 90.47619 | 92.10526 | 86.36364 | 94.59459 |
| 0.45 | 90.47619 | 94.73684 | 90.47619 | 94.73684 |
| 0.50 | 90.47619 | 94.73684 | 90.47619 | 94.73684 |

H) MMP7 + MUC2

| | | | | |
|---|---|---|---|---|
| 0.20 | 100.00000 | 78.94737 | 72.41379 | 100.00000 |
| 0.25 | 100.00000 | 81.57895 | 75.00000 | 100.00000 |
| 0.35 | 100.00000 | 86.84211 | 80.76923 | 100.00000 |
| 0.40 | 95.23810 | 86.84211 | 80.00000 | 97.05882 |
| 0.45 | 90.47619 | 86.84211 | 79.16667 | 94.28571 |
| 0.50 | 90.47619 | 92.10526 | 86.36364 | 94.59459 |

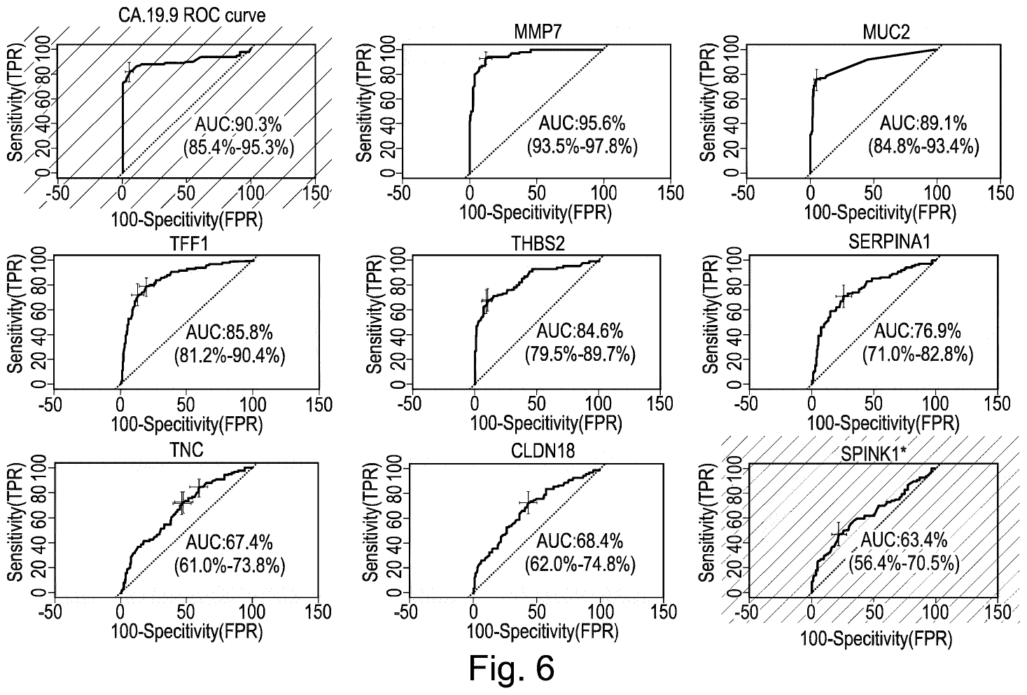

FIGS. 6A-D show examples of analysis performed on the ELISA data described above via log regression algorithm with backward elimination to differentiate between healthy and pancreatic cancer patients. The dataset was split into training and test sets (80% and 20% of data respectively). The analysis was performed by starting with all 20 variables (the different biomarkers in Table 1) and one by one variables were removed. Akaike Information Criterion (AIC) was monitored and maintained as low as possible to reduce overfitting and improve the model. Markers of interest were those that caused AIC to increase when the marker was removed. In certain examples, the process may be carried out a least 50 times to identify markers of interest. The resulting identified 7 biomarker combination of interest included MMP7, CA 19-9, MUC2, TNC, THBS2, TFF1, and REG1B unexpectedly provided a high AUC of 99.4%. The resulting identified 6 biomarker combination of interest included MMP7, MUC2, TNC, THBS2, TFF1, and REG1B with an unexpectedly high AUC of 98.5%. The resulting identified 5 biomarker combination of interest included MMP7, MUC2, TNC, THBS2, and TFF1 with an unexpectedly high AUC of 95.9%.

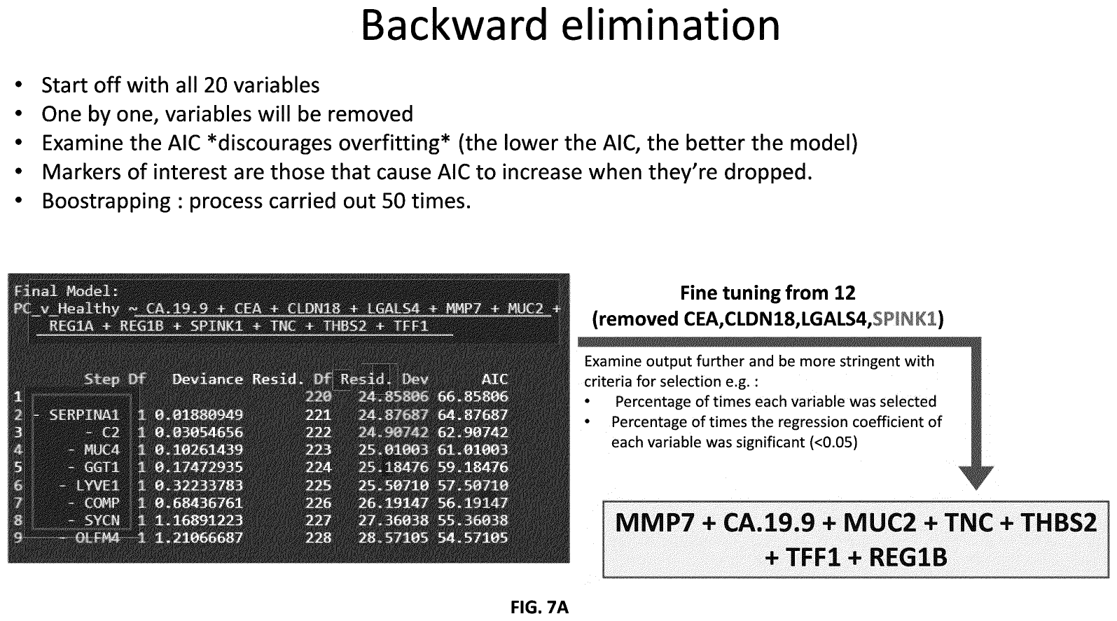

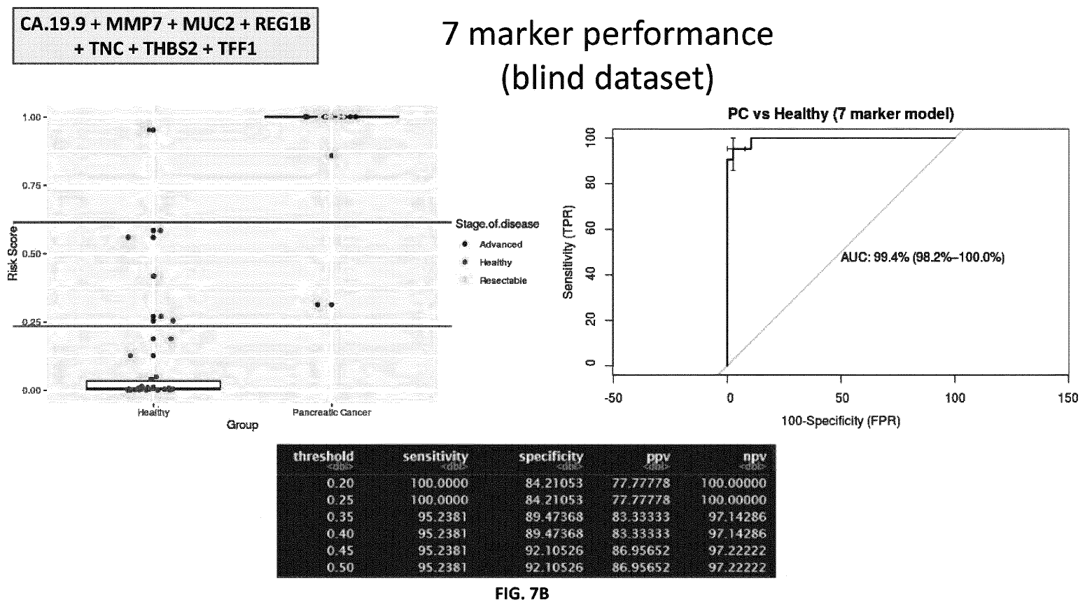

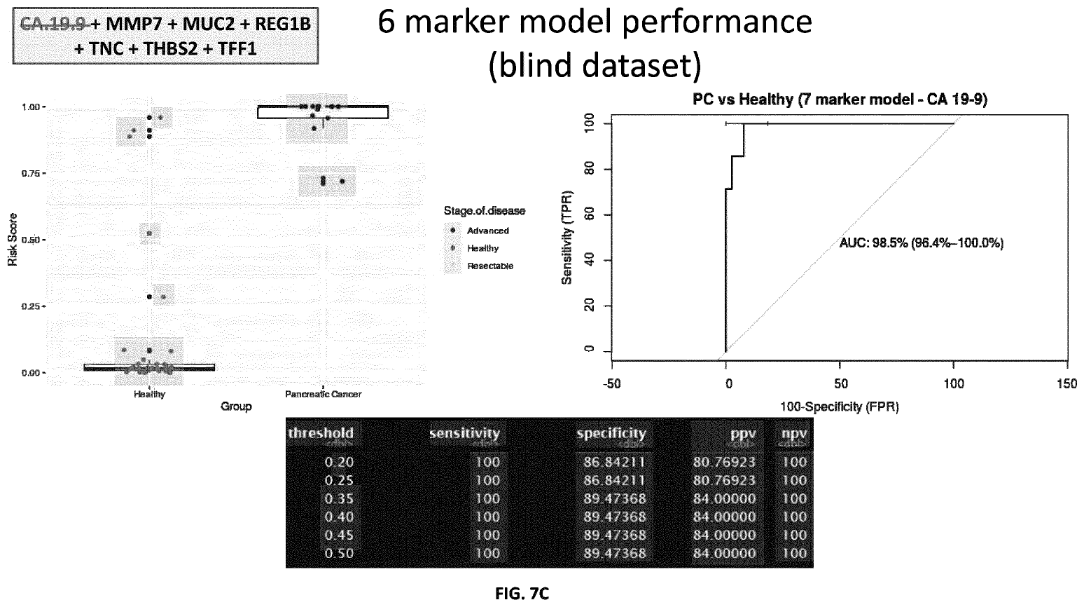

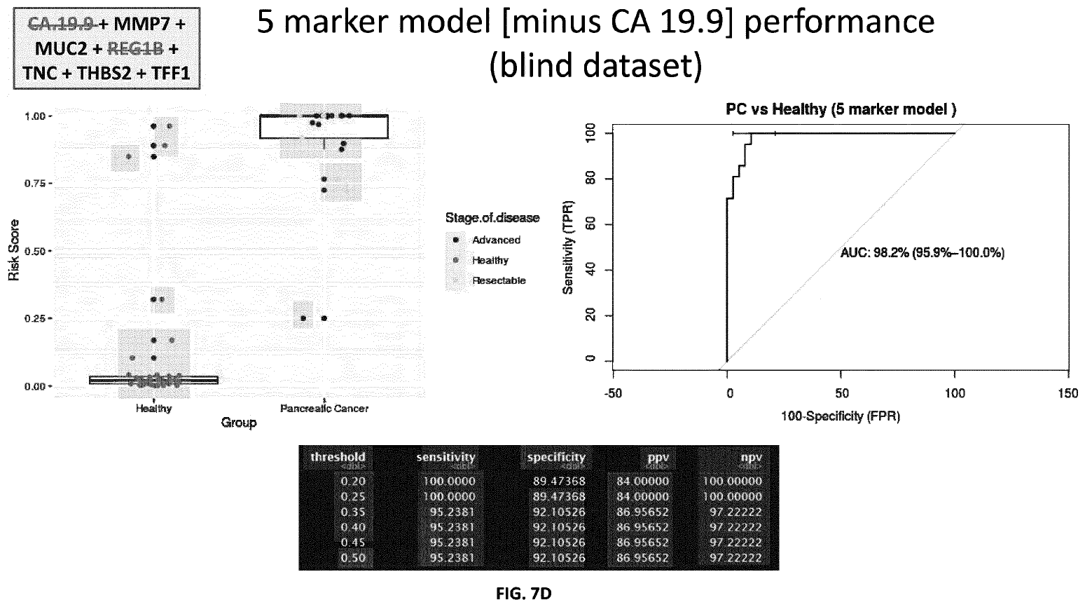

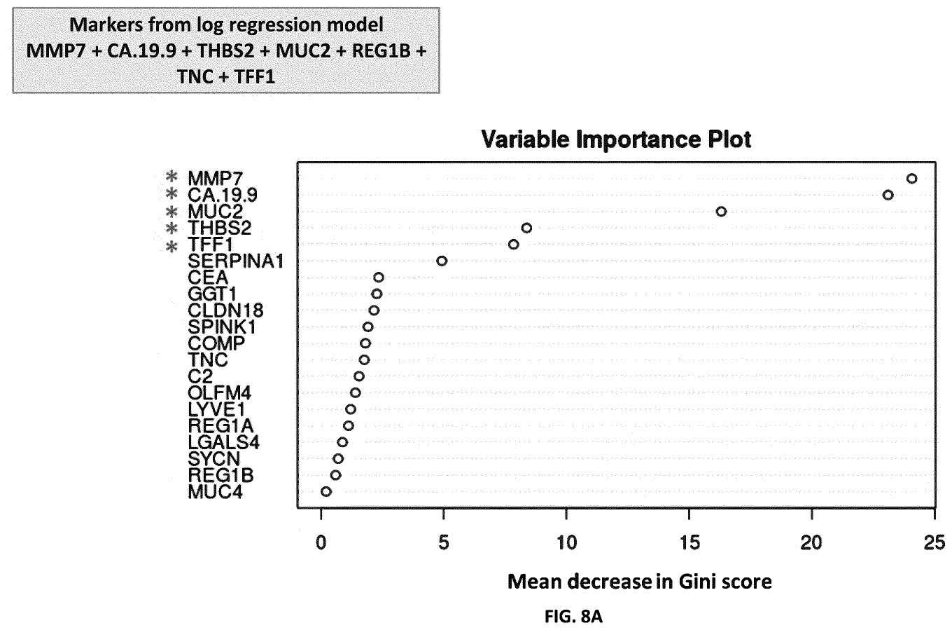

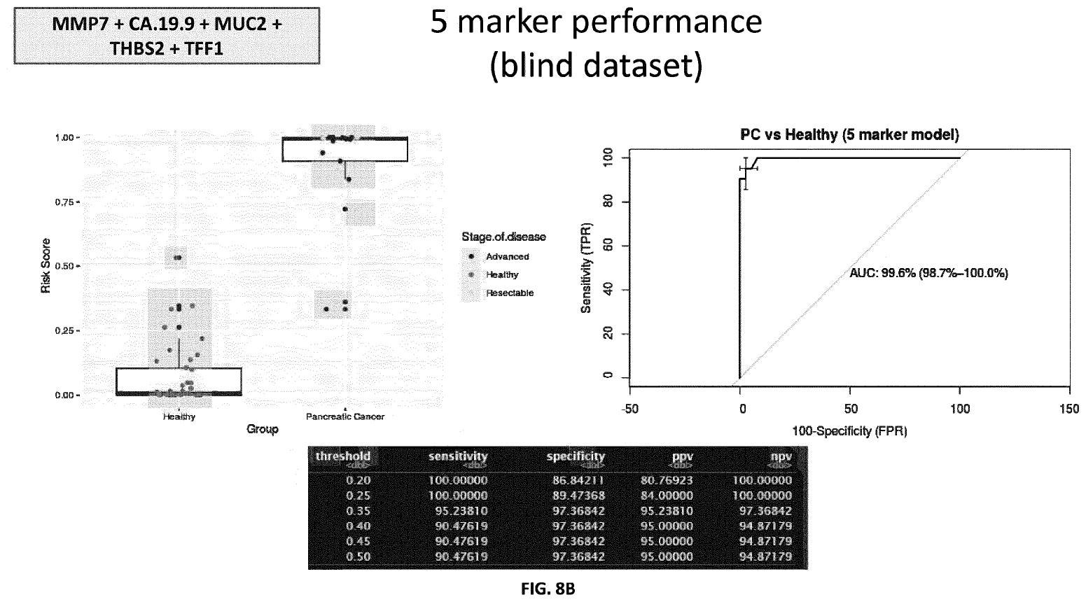

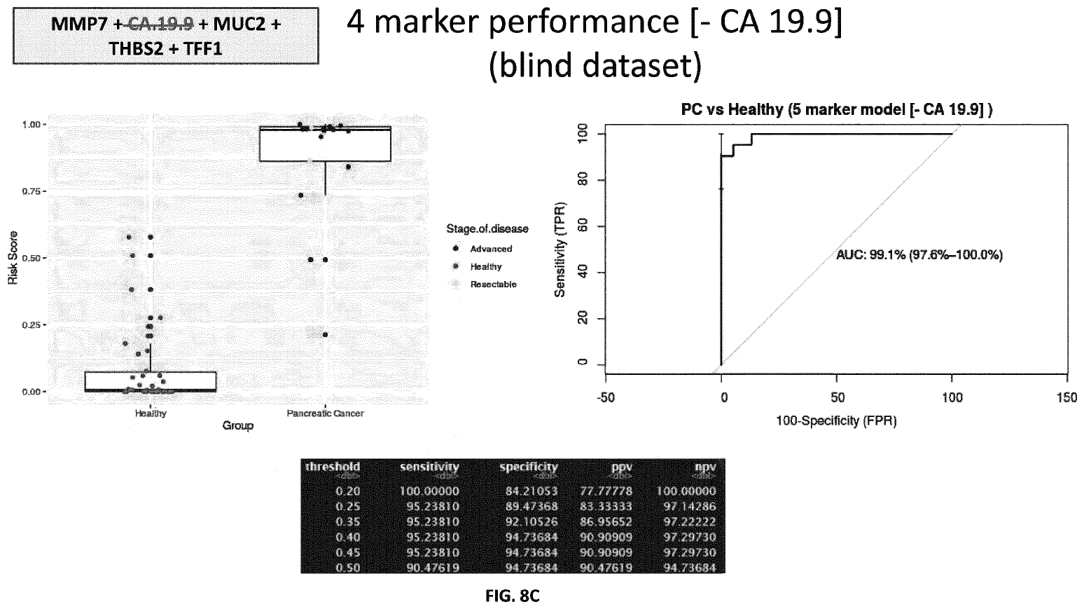

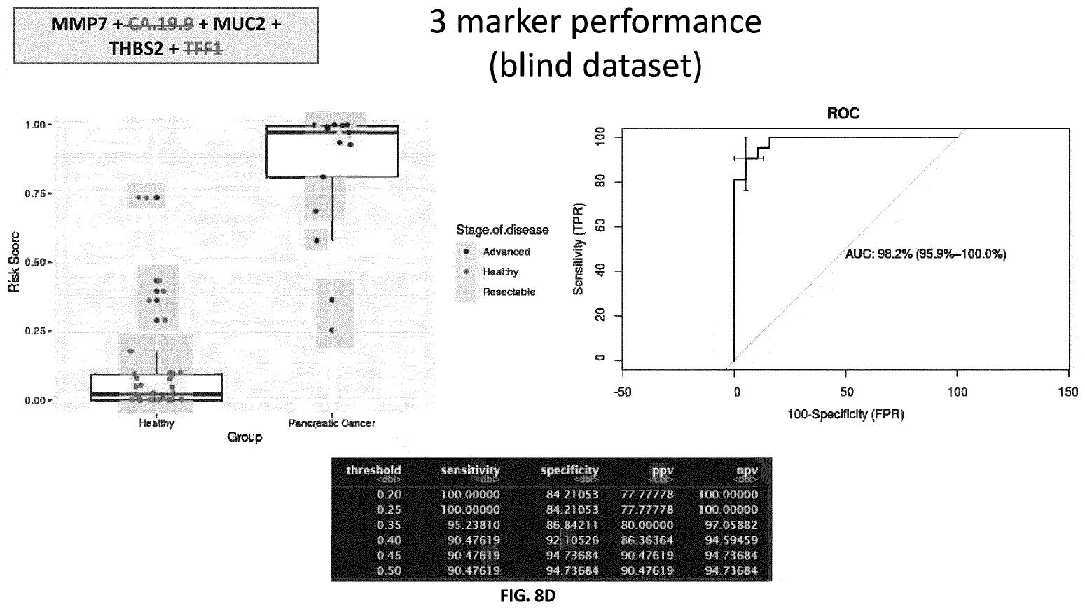

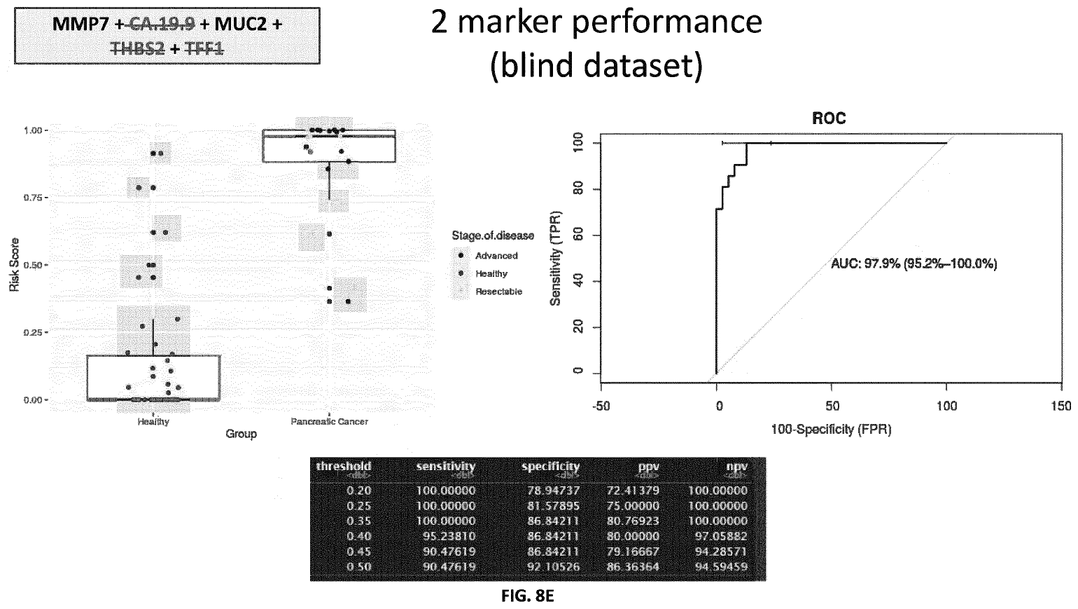

FIGS. 7A-7E show examples of analysis performed on the ELISA data described above via Random Forest classifier. As with the backward elimination, the dataset was split into training and test sets (80% and 20% of data respectively). First, a random subsample of the dataset was selected (training data only) and some set aside. Next, a random subset of all variables was chosen and the best performing variable made to be a “splitter variable,” which attempts to separate pancreatic cancer and healthy patients. The tree was then grown with additional ‘splitter variables’ until purely one class or the other is formed. The resulting tree was then “tested” against patients that were set aside in the beginning. Several decision trees may be constructed using techniques like there, for example 100s, 1000s, or 10000s of times. The resulting analysis identified 5 biomarker combination of interest included MMP7, CA 19-9, MUC2, THBS2, and TFF1 with an unexpectedly high AUC of 99.6%. The 4 biomarker combination of interest included MMP7, MUC2, THBS2, and TFF1 with an unexpectedly high AUC of 97.6%. The 3 biomarker combination of interest included MMP7, MUC2, and THBS2 with an unexpectedly high AUC of 98.2%. Lastly, the 2 biomarker combination of interest included MMP7 and MUC2 with an unexpectedly high AUC of 97.9%.

### Risk of Pancreatic Cancer

Disclosed herein are methods of determining if a subject has an increased risk of suffering from pancreatic cancer. The methods may comprise analyzing at least one serum, plasma, and/or whole blood (e.g. finger prick capillary blood) sample from the subject to determine a value for the presence of particular combinations of biomarkers and comparing the value of the subject's biomarkers with the biomarkers of a normal profile. A change in the values of the subject's biomarkers, over or under normal values may be indicative that the subject has an increased risk of suffering from pancreatic cancer compared to a normal individual.

As used herein, the term subject or “test subject” indicates a mammal, in particular a human or non-human primate. The test subject may or may not be in need of an assessment of a predisposition to pancreatic cancer. For example, the test subject may have a condition or may have been exposed to conditions that are associated with pancreatic cancer prior to applying the methods described herein. In another example, the test subject has not been identified as a subject that may have a condition or may have been exposed to injuries or conditions that are associated with pancreatic cancer prior to applying the methods and apparatuses disclosed herein.

As used herein, the term “increased risk” is used to mean that the test subject has an increased chance of developing or acquiring pancreatic cancer compared to a normal individual. The increased risk may be relative or absolute and may be expressed qualitatively or quantitatively. For example, an increased risk may be expressed as simply determining that the subject has a higher concentration of a particular combination of biomarkers and placing the patient in an “increased risk” category, based upon previous population studies. As used herein, examples of expressions of an increased risk include but are not limited to, odds, probability, odds ratio, p-values, attributable risk, relative frequency, positive predictive value, negative predictive value, and relative risk.

Increased risk can also be determined from p-values that are derived using logistic regression. Binomial (or binary) logistic regression is a form of regression which is used when the dependent is a dichotomy and the independents are of any type. Logistic regression can be used to predict a dependent variable on the basis of continuous and/or categorical independents and to determine the percent of variance in the dependent variable explained by the independents; to rank the relative importance of independents; to assess interaction effects; and to understand the impact of covariate control variables. Logistic regression applies maximum likelihood estimation after transforming the dependent into a “logit” variable (the natural log of the odds of the dependent occurring or not). In this way, logistic regression estimates the probability of a certain event occurring.

Examples of test samples or sources of components for the devices and methods described herein include, but are not limited to, biological fluids, which can be tested by suitable methods described herein, and include but are not limited to whole blood, such as but not limited to peripheral blood, serum, plasma, cerebrospinal fluid, urine, amniotic fluid, lymph fluids, and various external secretions of the respiratory, intestinal and genitourinary tracts, tears, saliva, milk, white blood cells, myelomas and the like. Test samples to be assayed also include but are not limited to tissue specimens including normal and abnormal tissue.

Techniques to assay levels of individual biomarkers from test samples are well known to the skilled technician, and the methods and apparatuses of this disclosure are is not limited by the means by which the components are assessed. In one example, levels of the individual components of the proteomic profile are assessed using mass spectrometry in conjunction with ultra-performance liquid chromatography (UPLC), high-performance liquid chromatography (HPLC), and UPLC to name a few. Other methods of assessing levels of the individual components include biological methods, such as but not limited to ELISA assays.

Samples taken from subjects may or may not processed prior to assaying. For example, whole blood may be taken from an individual and the blood sample may be processed, e.g., centrifuged, to isolate plasma or serum from the blood. The sample may or may not be stored, e.g., frozen, prior to processing or analysis.

The levels of depletion or augmentation of the combinations of biomarkers compared to normal levels can vary. In one example, the levels of any one or more of the proteins is at least 1.05, 1.1, 1.2, 1.3, 1.4, 1.5, 1.6, 1.7, 1.8, 1.9, 2, 3, 4, 5, 6, 7, 8, 9, 10, 11, 12, 13, 14, 15, 16, 17, 18, 19, 20, 25, 30, 35, 40, 45, 50, 55 or 60 times lower than normal levels. In another example, the levels of any one or more of the proteins is at least 1.05, 1.1, 1.2, 1.3, 1.4, 1.5, 1.6, 1.7, 1.8, 1.9, 2, 3, 4, 5, 6, 7, 8, 9, 10, 11, 12, 13, 14, 15, 16, 17, 18, 19, 20, 25, 30, 35, 40, 45, 50, 55 or 60 times higher than normal levels. The number of “times” the levels of a protein is lower or higher over normal can be a relative or absolute number of times. In the alternative, the levels of the proteins may be normalized to a standard and these normalized levels can then be compared to one another to determine if a protein is lower or higher.

Thus, the present disclosure also includes methods of monitoring the progression of pancreatic cancer in a subject, with the methods comprising testing the subject for one or more combinations of biomarkers indicator of pancreatic cancer more than once over a period of time. For example, some examples may include testing the subject two, three, four, five, six, seven, eight, nine, 10 or even more times over a period of time, such as a year, two years, three, years, four years, five years, six years, seven years, eight years, nine years or even 10 years or longer. The methods of monitoring a subject's risk of having pancreatic cancer would also include examples in which the subject is tested during and after treatment of pancreatic cancer. In other words, also disclosed are includes methods of monitoring the efficacy of treatment of proteomic impairment by assessing the presence of certain combinations of biomarkers over the course of the treatment and after the treatment.

### Terminology

At least some of the processes, methods, and algorithms described in the preceding sections may be embodied in, and fully or partially automated by, code modules executed by one or more computer systems or computer processors comprising computer hardware. The code modules may be stored on any type of non-transitory computer-readable medium or computer storage device, such as hard drives, solid state memory, optical disc, and/or the like. The systems and modules may also be transmitted as generated data signals (for example, as part of a carrier wave or other analog or digital propagated signal) on a variety of computer-readable transmission mediums, including wireless-based and wired/cable-based mediums, and may take a variety of forms (for example, as part of a single or multiplexed analog signal, or as multiple discrete digital packets or frames). The processes and algorithms may be implemented partially or wholly in application-specific circuitry. The results of the disclosed processes and process steps may be stored, persistently or otherwise, in any type of non-transitory computer storage such as, for example, volatile or non-volatile storage.

The various features and processes described above may be used independently of one another, or may be combined in various ways. All possible combinations and subcombinations are intended to fall within the scope of this disclosure. In addition, certain method or process blocks may be omitted in some implementations. The methods and processes described herein are also not limited to any particular sequence, and the blocks or states relating thereto can be performed in other sequences that are appropriate. For example, described blocks or states may be performed in an order other than that specifically disclosed, or multiple blocks or states may be combined in a single block or state. The example blocks or states may be performed in serial, in parallel, or in some other manner. Blocks or states may be added to or removed from the disclosed example embodiments. The example systems and components described herein may be configured differently than described. For example, elements may be added to, removed from, or rearranged compared to the disclosed example embodiments.

Conditional language, such as, among others, “can,” “could,” “might,” or “may,” unless specifically stated otherwise, or otherwise understood within the context as used, is generally intended to convey that certain embodiments include, while other embodiments do not include, certain features, elements and/or steps. Thus, such conditional language is not generally intended to imply that features, elements and/or steps are in any way required for one or more embodiments or that one or more embodiments necessarily include logic for deciding, with or without user input or prompting, whether these features, elements and/or steps are included or are to be performed in any particular embodiment. The term “including” means “included but not limited to.” The term “or” means “and/or.”

Any process descriptions, elements, or blocks in the flow or block diagrams described herein and/or depicted in the attached figures should be understood as potentially representing modules, segments, or portions of code which include one or more executable instructions for implementing specific logical functions or steps in the process. Alternate implementations are included within the scope of the embodiments described herein in which elements or functions may be deleted, executed out of order from that shown or discussed, including substantially concurrently or in reverse order, depending on the functionality involved, as would be understood by those skilled in the art.

All of the methods and processes described above may be at least partially embodied in, and partially or fully automated via, software code modules executed by one or more computers. For example, the methods described herein may be performed by the computing system and/or any other suitable computing device. The methods may be executed on the computing devices in response to execution of software instructions or other executable code read from a tangible computer readable medium. A tangible computer readable medium is a data storage device that can store data that is readable by a computer system. Examples of computer readable mediums include read-only memory, random-access memory, other volatile or non-volatile memory devices, CD-ROMs, magnetic tape, flash drives, and optical data storage devices.

It should be emphasized that many variations and modifications may be made to the above-described embodiments, the elements of which are to be understood as being among other acceptable examples. All such modifications and variations are intended to be included herein within the scope of this disclosure. The foregoing description details certain embodiments. It will be appreciated, however, that no matter how detailed the foregoing appears in text, the systems and methods can be practiced in many ways. For example, a feature of one embodiment may be used with a feature in a different embodiment. As is also stated above, it should be noted that the use of particular terminology when describing certain features or aspects of the systems and methods should not be taken to imply that the terminology is being re-defined herein to be restricted to including any specific characteristics of the features or aspects of the systems and methods with which that terminology is associated.

Various embodiments of a pancreatic cancer detection device, method, and system are disclosed herein. These various embodiments may be used alone or in combination, and various changes to individual features of the embodiments may be altered, without departing from the scope of the invention. For example, the order of various method steps may in some instances be changed, and/or one or more optional features may be added to or eliminated from a described device. Therefore, the description of the embodiments provided above should not be interpreted as unduly limiting the scope of the invention as it is set forth in the claims.

Various modifications to the implementations described in this disclosure may be made, and the generic principles defined herein may be applied to other implementations without departing from the spirit or scope of this disclosure. Thus, the scope of the disclosure is not intended to be limited to the implementations shown herein, but are to be accorded the widest scope consistent with this disclosure, the principles and the novel features disclosed herein.

Certain features that are described in this specification in the context of separate embodiments also can be implemented in combination in a single embodiment. Conversely, various features that are described in the context of a single embodiment also can be implemented in multiple embodiments separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations are depicted in the drawings and/or the specification in a particular order, such operations need not be performed in the particular order shown or in sequential order, or that all illustrated operations be performed, to achieve desirable results. Further, the drawings may schematically depict one more example processes in the form of a flow diagram. However, other operations that are not depicted can be incorporated in the example processes that are schematically illustrated. For example, one or more additional operations can be performed before, after, simultaneously, or between any of the illustrated operations. Moreover, the separation of various system components in the embodiments described above should not be interpreted as requiring such separation in all embodiments. Additionally, other embodiments are within the scope of the following claims. In some cases, the actions recited in the claims can be performed in a different order and still achieve desirable results.

While the present description sets forth specific details of various examples, it will be appreciated that the description is illustrative only and should not be construed in any way as limiting. Furthermore, various applications of such examples and modifications thereto, which may occur to those who are skilled in the art, are also encompassed by the general concepts described herein. Each and every feature described herein, and each and every combination of two or more of such features, is included within the scope of the present invention provided that the features included in such a combination are not mutually inconsistent.

All figures, tables, and appendices, as well as patents, applications, and publications, referred to above, are hereby incorporated by reference.

Some examples have been described in connection with the accompanying drawings. However, it should be understood that the figures are not drawn to scale. Distances, angles, etc. are merely illustrative and do not necessarily bear an exact relationship to actual dimensions and layout of the devices illustrated. Components can be added, removed, and/or rearranged. Further, the disclosure herein of any particular feature, aspect, method, property, characteristic, quality, attribute, element, or the like in connection with various examples can be used in all other examples set forth herein. Additionally, it will be recognized that any methods described herein may be practiced using any device suitable for performing the recited steps.

For purposes of this disclosure, certain aspects, advantages, and novel features are described herein. It is to be understood that not necessarily all such advantages may be achieved in accordance with any particular example. Thus, for example, those skilled in the art will recognize that the disclosure may be embodied or carried out in a manner that achieves one advantage or a group of advantages as taught herein without necessarily achieving other advantages as may be taught or suggested herein.

Although these inventions have been disclosed in the context of certain preferred examples and examples, it will be understood by those skilled in the art that the present inventions extend beyond the specifically disclosed examples to other alternative examples and/or uses of the inventions and obvious modifications and equivalents thereof. In addition, while several variations of the inventions have been shown and described in detail, other modifications, which are within the scope of these inventions, will be readily apparent to those of skill in the art based upon this disclosure. It is also contemplated that various combination or sub-combinations of the specific features and aspects of the examples may be made and still fall within the scope of the inventions. It should be understood that various features and aspects of the disclosed examples can be combined with or substituted for one another in order to form varying modes of the disclosed inventions. Further, the actions of the disclosed processes and methods may be modified in any manner, including by reordering actions and/or inserting additional actions and/or deleting actions. Thus, it is intended that the scope of at least some of the present inventions herein disclosed should not be limited by the particular disclosed examples described above. The limitations in the claims are to be interpreted broadly based on the language employed in the claims and not limited to the examples described in the present specification or during the prosecution of the application, which examples are to be construed as non-exclusive.

1. A pancreatic cancer detection device, comprising:
a solid surface comprising a plurality of antibodies bound to the solid surface, the solid surface configured to indicate selective binding between each antibody and a corresponding target protein; and
wherein the antibodies are configured to selectively bind to two of the target proteins selected from the group consisting of MMP7, CA 19-9, THBS2, MUC2, REG1B, TNC, and TFF1.

2. The pancreatic cancer detection device of claim 1, wherein the solid surface comprises antibodies configured to selectively bind three target proteins selected from the group.

3. The pancreatic cancer detection device of claim 1, wherein the solid surface comprises antibodies configured to selectively bind four target proteins selected from the group.

4. The pancreatic cancer detection device of claim 1, wherein the solid surface comprises antibodies configured to selectively bind five target proteins selected from the group.

5. The pancreatic cancer detection device of claim 1, wherein the solid surface comprises antibodies configured to selectively bind six target proteins selected from the group.

6. The pancreatic cancer detection device of claim 1, wherein the solid surface comprises antibodies configured to selectively bind seven target proteins selected from the group.

7. The pancreatic cancer detection device of claim 1, wherein the solid surface comprises antibodies configured to selectively bind MMP7, CA 19-9, MUC2, THBS2, and TFF1.

8. The pancreatic cancer detection device of claim 1, wherein the solid surface comprises antibodies configured to selectively bind MMP7, CA 19-9, TNC, TIIBS2, and TFF1.

9. The pancreatic cancer detection device of claim 1, wherein the solid surface comprises antibodies configured to selectively bind MMP7, MUC2, TNC, THBS2, and TFF1.

10. The pancreatic cancer detection device of claim 1, wherein the solid surface comprises antibodies configured to selectively bind MMP7, MUC2, REG1B, TNC, THBS2, and TFF1.

11. The pancreatic cancer detection device of any one of the previous claims, wherein the lateral flow detection surface comprises a lateral flow detection test trip.

12. The pancreatic cancer detection device of claim 11, further comprising a control line.

13. The pancreatic cancer detection devices of claim 1, wherein indicating comprises visually indicating binding to a user.

14. A method of detecting pancreatic cancer, comprising:
collecting a biological sample from a subject;
contacting the biological sample with the pancreatic detection device of claim 1; and
indicating a likelihood of an incidence of pancreatic cancer in the subject.

15. The method of claim 14, wherein the biological sample comprises whole blood.

16. The method of claim 14, wherein the biological sample comprises serum.

17. The method of claim 14, further comprising providing a report, the report indicating the likelihood of an incidence of pancreatic cancer in a subject.
